(19) United States
(12) Patent Application Publication
(10) Pub. No.: US 2023/0127329 A1
(43) Pub. Date: Apr. 27, 2023

(54) LACTONE COMPOUND

(71) Applicant: FUJIFILM Corporation, Tokyo (JP)

(72) Inventors: Kazufumi OMURA, Kanagawa (JP); Noriyuki KATO, Tokyo (JP)

(73) Assignee: FUJIFILM Corporation, Tokyo (JP)

(21) Appl. No.: 18/047,655

(22) Filed: Oct. 19, 2022

(30) Foreign Application Priority Data
Oct. 22, 2021 (JP) 2021-172846

(51) Int. Cl. C07D 407/12

(52) U.S. Cl. CPC C07D 407/12

(57) ABSTRACT

An object of the present invention is to provide a lactone compound having excellent radical trapping performance.

Provided is a lactone compound represented by Formula (I).

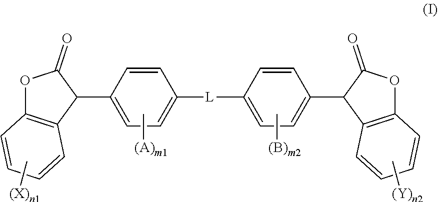

L represents a linking group represented by Formula (L1) or Formula (L2).

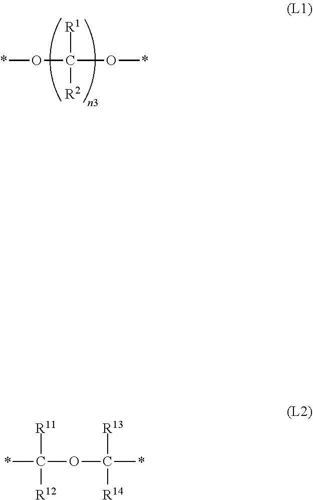

### CROSS-REFERENCE TO RELATED APPLICATIONS

The present application claims priority under 35 U.S.C. § 119 to Japanese Patent Application No. 2021-172846, filed on Oct. 22, 2021. The above application is hereby expressly incorporated by reference, in its entirety, into the present application.

### BACKGROUND OF THE INVENTION

### 1. Field of the Invention

The present invention relates to a novel lactone compound.

### 2. Description of the Related Art

3-Arylbenzofuranone, which is a kind of a lactone compound, has been known to be suitable as a stabilizer for an organic material susceptible to oxidation, heat, or photodecomposition (for example, see JP1995-233160A (JP-H07-233160A) and JP1995-165745A (JP-H07-165745A)).

### SUMMARY OF THE INVENTION

As a result of studying on the lactone compounds disclosed in JP1995-233160A (JP-H07-233160A) and JP1995-165745A (JP-H07-165745A), the present inventors have found that there is room for improvement in performance to trap radicals (hereinafter, abbreviated as a “radical trapping performance”).

An object of the present invention is to provide a lactone compound having excellent radical trapping performance.

As a result of intensive studies to achieve the above-described object, the present inventors have found that a bis-type lactone compound having a predetermined linking group has excellent radical trapping performance, and have completed the present invention.

That is, the present inventors have found that the above-described object can be achieved by adopting the following configurations.
[1] A lactone compound represented by Formula (I) described later.
[2] The lactone compound according to [1], in which both m1 and m2 in Formula (I) described later represent 0.
[3] The lactone compound according to [1] or [2], in which both n1 and n2 in Formula (I) described later represent 2.
[4] The lactone compound according to [3], in which the lactone compound is represented by Formula (I-1) described later.

According to the present invention, it is possible to provide a lactone compound having excellent radical trapping performance.

### DESCRIPTION OF THE PREFERRED EMBODIMENTS

Hereinafter, the present invention will be described in detail.

The description of configuration requirements described below may be made on the basis of representative embodiments of the present invention, but it should not be construed that the present invention is limited to those embodiments.

In the specification of the present application, the numerical range expressed by using “to” means a range including the numerical values before and after “to” as the lower limit value and the upper limit value.

Lactone Compound

A lactone compound according to an embodiment of the present invention is a lactone compound represented by Formula (I).

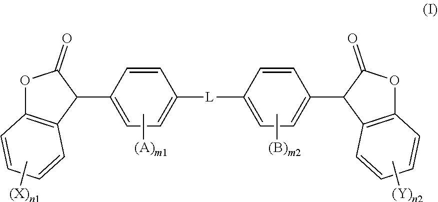

Here, in Formula (I),

A, B, X, and Y each independently represent an alkyl group or an alkoxy group,

m1 represents an integer of 0 to 4, and in a case where m1 is an integer of 2 to 4, a plurality of A's may be the same substituent or substituents different from each other,

m2 represents an integer of 0 to 4, and in a case where m2 is an integer of 2 to 4, a plurality of B's may be the same substituent or substituents different from each other,

n1 represents an integer of 0 to 4, and in a case where n1 is an integer of 2 to 4, a plurality of X's may be the same substituent or substituents different from each other and may be bonded to each other to form a ring,

n2 represents an integer of 0 to 4, and in a case where n2 is an integer of 2 to 4, a plurality of Y's may be the same substituent or substituents different from each other, and may be bonded to each other to form a ring, and

L represents a linking group represented by Formula (L1) or Formula (L2).

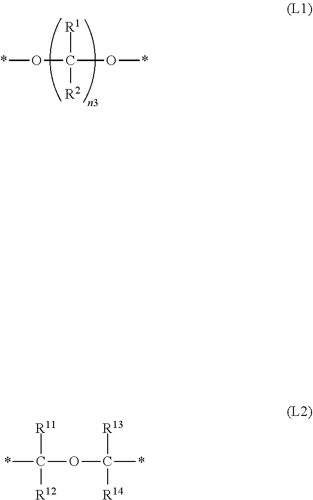

Here, in Formulae (L1) and (L2), * represents a bonding position to a phenylene group in Formula (I).

In Formula (L1), R¹ and R² each independently represent a hydrogen atom, an alkyl group, or an alkoxy group, n3 represents an integer of 1 to 12, and in a case where n3 is an integer of 2 to 12, a plurality of R's may be the same substituent or substituents different from each other, and a plurality of R²'s may be the same substituent or substituents different from each other.

In Formula (L2), R¹¹, R¹², R¹³, and R¹⁴ each independently represent a hydrogen atom, an alkyl group, or an alkoxy group.

Hereinafter, A, B, X, Y, m1, m2, n1, n2, and L in Formula (I) will be described in detail.

In Formula (I), as the alkyl group represented by one aspect of A and B, for example, a linear, branched, or cyclic alkyl group having 1 to 18 carbon atoms is preferable, an alkyl group having 1 to 8 carbon atoms (for example, a methyl group, an ethyl group, a propyl group, an isopropyl group, an n-butyl group, an isobutyl group, a sec-butyl group, a tert-butyl group, a cyclohexyl group, and the like) is more preferable, an alkyl group having 1 to 4 carbon atoms is still more preferable, and a methyl group or an ethyl group is particularly preferable.

In addition, as the alkoxy group represented by one aspect of A and B, for example, an alkoxy group having 1 to 18 carbon atoms is preferable, an alkoxy group having 1 to 8 carbon atoms (for example, a methoxy group, an ethoxy group, an n-butoxy group, a methoxyethoxy group, and the like) is more preferable, an alkoxy group having 1 to 4 carbon atoms is still more preferable, and a methoxy group or an ethoxy group is particularly preferable.

both m1 and m2 in Formula (I) represent an integer of 0 to 4, and it is preferable that both represent an integer of 0 to 2. In addition, from the reason that the molecule is rigid and it is difficult to bleed in a case of being blended with a polymer compound, it is more preferable that both represent 0.

In Formula (I), examples of the alkyl group represented by one aspect of X and Y include the same alkyl group as the alkyl group represented by one aspect of A and B described above.

In addition, examples of the alkoxy group represented by one aspect of X and Y include the same alkoxy group as the alkoxy group represented by one aspect of A and B described above.

Among these, X and Y are preferably a branched alkyl group, more preferably a branched alkyl group having 3 to 12 carbon atoms, and still more preferably a tert-butyl group, a tert-amyl group (1,1-dimethylpropyl group), or a tert-octyl group (1,1,3,3-tetramethylbutyl group).

On the other hand, in a case where a plurality of X's are bonded to each other to form a ring and a case where a plurality of Y's are bonded to each other to form a ring, examples of the ring to be formed include a benzene ring, a naphthalene ring, and a cyclohexane ring, and among them, a benzene ring is preferable.

both n1 and n2 in Formula (I) represent an integer of 0 to 4, and it is preferable that both represent an integer of 1 to 4. In addition, in the synthesis using the reaction of phenol and glyoxylic acid (Friedel-Crafts reaction), from the reason that reactivity for forming a lactone ring including the substituent X or Y is good, it is more preferable that both represent 2.

As described above, L in Formula (I) represents a linking group represented by Formula (L1) or Formula (L2).

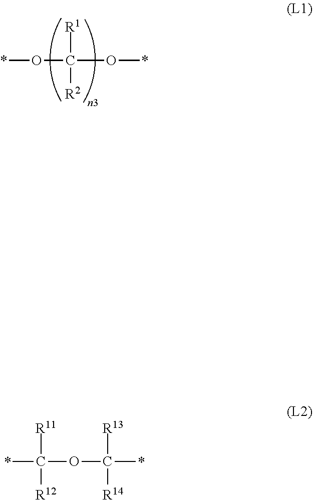

Here, in Formulae (L1) and (L2), * represents a bonding position to a phenylene group in Formula (I).

In Formula (L1), R¹ and R² each independently represent a hydrogen atom, an alkyl group, or an alkoxy group, n3 represents an integer of 1 to 12, and in a case where n3 is an integer of 2 to 12, a plurality of R's may be the same substituent or substituents different from each other, and a plurality of R²'s may be the same substituent or substituents different from each other.

In Formula (L2), R¹¹, R¹², R¹³, and R¹⁴ each independently represent a hydrogen atom, an alkyl group, or an alkoxy group.

Examples of the alkyl group represented by one aspect of R¹ and R² in Formula (L1) and R¹¹, R¹², R¹³, and R¹⁴ in Formula (L2) include the same alkyl group as the alkyl group represented by one aspect of A and B described above.

In addition, examples of the alkoxy group represented by one aspect of R¹ and R², and R¹¹, R¹², R¹³, and R¹⁴ include the same alkoxy group as the alkoxy group represented by one aspect of A and B described above.

In addition, n3 in Formula (L1) represents an integer of 1 to 12, and from the viewpoint of radical trapping performance, preferably represents an integer of 1 to 8, more preferably represents an integer of 1 to 4, and still more preferably represents 2.

In the synthesis using the reaction of phenol and glyoxylic acid (Friedel-Crafts reaction), from the reason that the reactivity for forming a lactone ring including the substituent X or Y is improved or the radical trapping performance is further improved, the lactone compound according to the embodiment of the present invention is preferably a lactone compound represented by Formula (I-1).

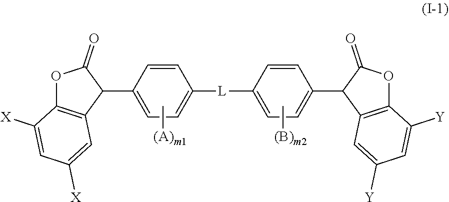

Here, A, B, X, Y, m1, m2, and L in Formula (I-1) are all the same as those described in Formula (I).

In the present invention, from the viewpoint of further improving the radical trapping performance, L in Formula (I) or Formula (I-1) preferably represents the linking group represented by Formula (L1), more preferably represents a linking group in which both R¹ and R² in Formula (L1) are hydrogen atoms, and still more preferably represents a linking group in which n3 in Formula (L1) is 2.

Specific examples of the lactone compound represented by Formula (I) include compounds (1) to (17) represented by Formulae (1) to (17).

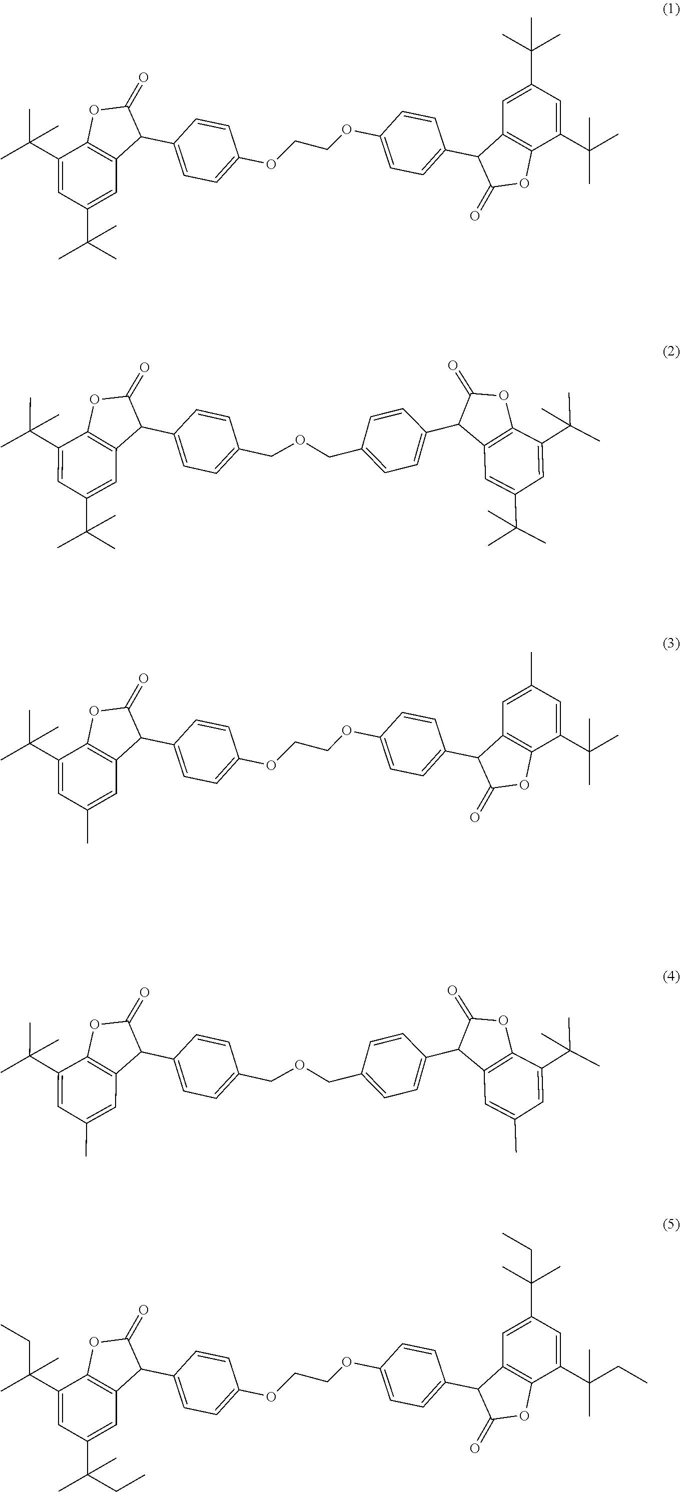

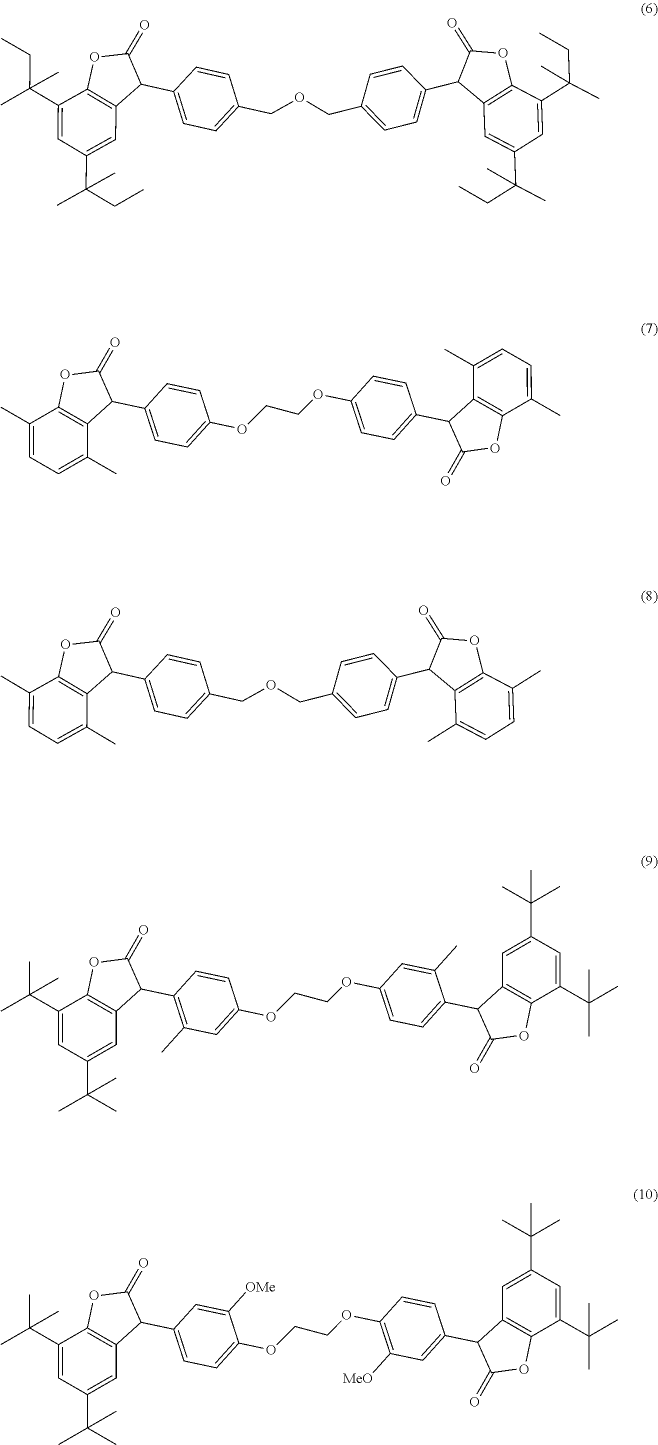

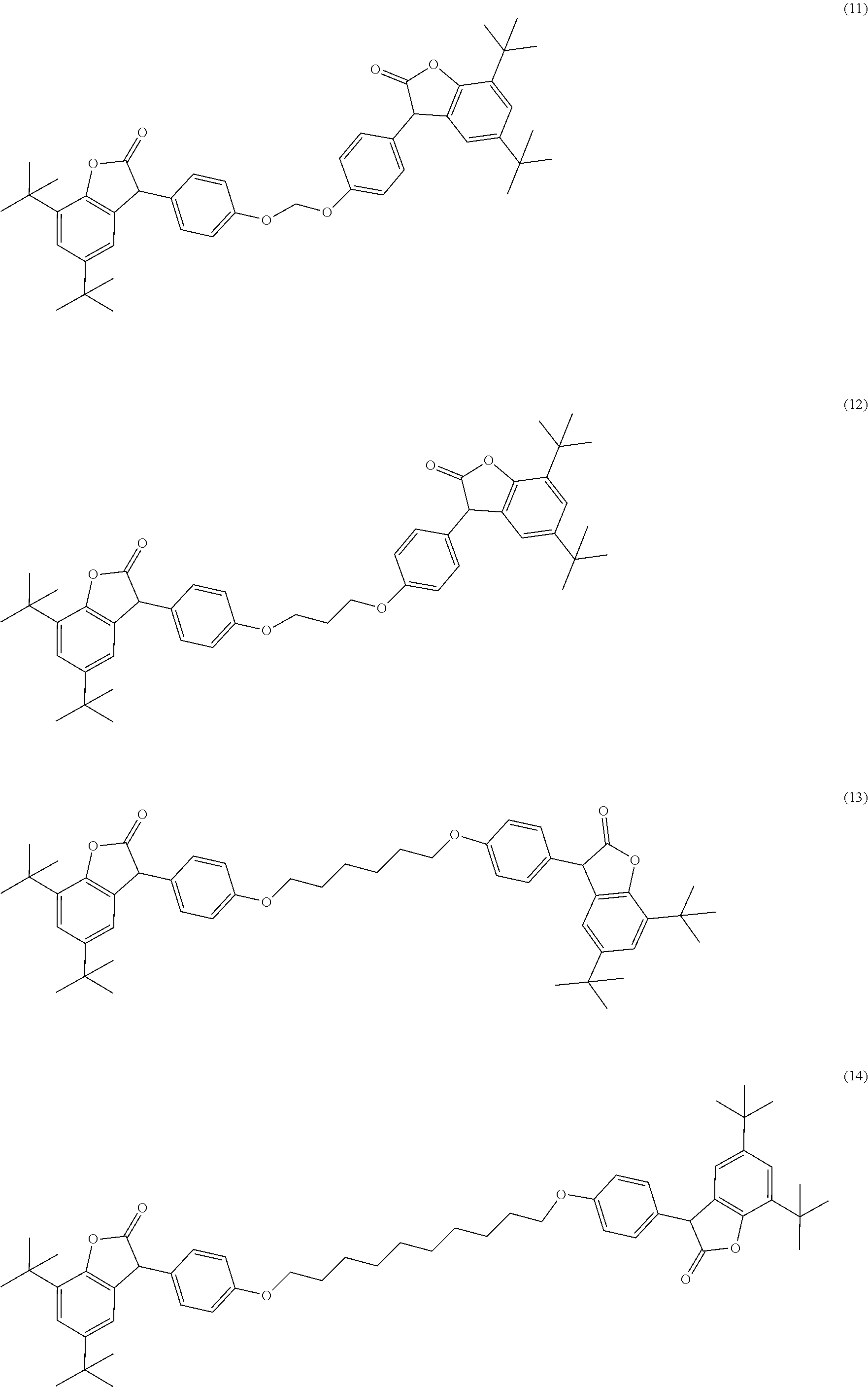

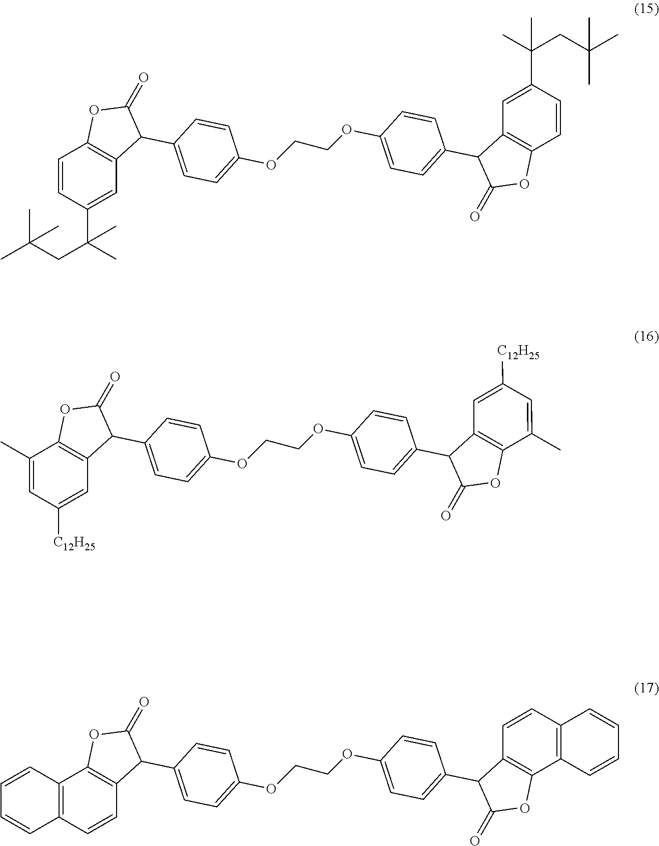

Polymer Compound

The lactone compound according to the embodiment of the present invention can be used as a stabilizer for a polymer compound.

Here, the polymer compound is not particularly limited as long as it corresponds to the organic material susceptible to oxidation, heat, or photodecomposition, and may be any of a water-soluble polymer compound or a water-insoluble polymer compound.

The water-soluble polymer compound is not particularly limited, and a known compound can be used. Specific examples thereof include proteins such as gelatin, casein, and albumin, polysaccharides such as starch and dextrin, cellulose and a derivative thereof (for example, carboxyl methyl cellulose, hydroxyl propyl cellulose, methyl cellulose, and the like), alginic acid, carrageenan, guar gum, xanthan gum, fucoidan, chitosan, hyaluronic acid, polyethylene oxide, polyvinyl alcohol, polyvinylpyrrolidone, polyvinyl methyl ether, polyacrylamide, polyethyleneimine, polyallylamine, polyvinylamine, polylysine, polyacrylic acid, and a graft-polymerized polymer thereof. In addition, a compound modified by a known method, such as succinated gelatin, can also be used.

The water-insoluble polymer compound is not particularly limited, and a known homopolymer or copolymer can be used.

Examples of the homopolymer include polymers such as vinyl acetate, vinyl chloride, styrene, methyl acrylate, butyl acrylate, methacrylonitrile, butadiene, and isoprene.

Examples of the copolymer include an ethylene/butadiene copolymer, a styrene/butadiene copolymer, a styrene/p-methoxystyrene copolymer, a styrene/vinyl acetate copolymer, a vinyl acetate/vinyl chloride copolymer, a vinyl acetate/diethyl maleate copolymer, a methyl methacrylate/acrylonitrile copolymer, a methyl methacrylate/butadiene copolymer, a methyl methacrylate/styrene copolymer, a methyl methacrylate/vinyl acetate copolymer, a methyl methacrylate/vinylidene chloride copolymer, a methyl acrylate/acrylonitrile copolymer, a methyl acrylate/butadiene copolymer, a methyl acrylate/styrene copolymer, a methyl acrylate/vinyl acetate copolymer, an acrylic acid/butyl acrylate copolymer, a methyl acrylate/vinyl chloride copolymer, a butyl acrylate/styrene copolymer, polyester, polycarbonate, and various urethanes.

A content of such a polymer compound is not particularly limited, but with respect to 100 parts by mass of the above-described lactone compound according to the embodiment of the present invention, is preferably 1,000 to 10,000,000 parts by mass, more preferably 100,000 to 10,000,000 parts by mass, and still more preferably 200,000 to 2,000,000 parts by mass.

In addition, with respect to 100 parts by mass of the polymer compound, a content of the above-described lactone compound according to the embodiment of the present invention is preferably 0.001 to 10 parts by mass, more preferably 0.001 to 1 parts by mass, still more preferably 0.001 to 0.1 parts by mass, and particularly preferably 0.005 to 0.05 parts by mass.

In addition, in a case where the lactone compound according to the embodiment of the present invention is used as a stabilizer or the like, co-stabilizers described in paragraphs [0180] to [0212] of JP1995-233160A (JP-H07-233160A) and paragraphs [0199] to [0222] of JP2019-014826A can be used in combination.

### EXAMPLES

Hereinafter, the present invention will be described in more detail with reference to examples. Materials, amounts used, ratios, treatment contents, treatment procedures, and the like shown in the following examples can be appropriately changed without departing from the spirit of the present invention. Accordingly, the scope of the present invention is not limited to the following Examples.

### Example 1

### Synthesis of Compound (1)

21.3 parts by mass of 2,4-di-tert-butylphenol (manufactured by Tokyo Chemical Industry Co., Ltd.), 10.4 parts by mass of glyoxylic acid monohydrate (manufactured by FUJIFILM Wako Pure Chemical Corporation), 0.051 parts by mass of p-toluenesulfonic acid monohydrate (manufactured by Tokyo Chemical Industry Co., Ltd.), and 40 parts by mass of 1,2-dichloroethane (manufactured by Tokyo Chemical Industry Co., Ltd.) were weighed in a flask.

Next, the weighed flask was placed in an oil bath at 105° C., and while distilling off the distillate using a Dean Stark tube, heating was continued until the internal temperature reached 86° C. by adding the same amount of 1,2-dichloroethane as the amount of distillation.

Next, the oil bath was heated to 120° C., and the resulting mixture was concentrated so that the residual amount of 1,2-dichloroethane was less than 10 parts by mass. Thereafter, the reaction solution was cooled to room temperature (23° C.), 100 parts by mass of hexane and 100 parts by mass of water were added thereto, and the mixture was stirred. Thereafter, the hexane layer was recovered, 100 parts by mass of saturated saline was added thereto, and the mixture was stirred. Thereafter, the hexane layer was recovered, 1 part by mass of magnesium sulfate (manufactured by FUJIFILM Wako Pure Chemical Corporation) was added thereto, and the mixture was dried for 1 hour.

Next, the magnesium sulfate was filtered off, and the hexane layer was concentrated to dryness using an evaporator to obtain 27.0 parts by mass of a brown viscous substance (1) containing a compound represented by Formula (1a).

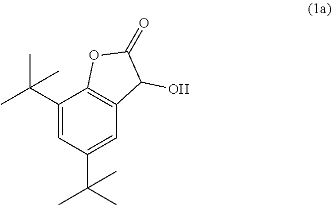 [SMILES: CC(C)(C)c1cc2c(c(C(C)(C)C)c1)OC(=O)C2O]

Next, in the brown viscous substance (1), 4.41 parts by mass of ethylene glycol diphenyl ether (manufactured by Tokyo Chemical Industry Co., Ltd.), 15.88 parts by mass of tin (IV) chloride pentahydrate (manufactured by Tokyo Chemical Industry Co., Ltd.), and 81 parts by mass of 1,2-dichloroethane (manufactured by Tokyo Chemical Industry Co., Ltd.) were weighed, and the mixture was refluxed for 4 hours using an oil bath at 105° C.

Next, after distilling off 40 parts by mass of the 1,2-dichloroethane, the reaction solution was cooled to room temperature (23° C.), and 200 parts by mass of ethyl acetate and 200 parts by mass of water were added thereto. Thereafter, the ethyl acetate layer was recovered, 200 parts by mass of saturated saline was added thereto, and the mixture was stirred. Thereafter, the ethyl acetate layer was recovered, and using 200 parts by mass of saturated saline, liquid separation purification was repeated until the pH of the saturated saline layer was to be 6.

Thereafter, the ethyl acetate layer was recovered, 1 part by mass of magnesium sulfate was added thereto, and the mixture was dried for 1 hour.

Next, the magnesium sulfate was filtered off through Celite, and the ethyl acetate layer was concentrated to dryness using an evaporator to obtain 21 parts by mass of a brown viscous substance (2).

The obtained brown viscous substance (2) was purified by column chromatography to obtain a compound (1) represented by Formula (1) described above.

¹H-nuclear magnetic resonance (NMR) data of the obtained compound (1) and assignments thereof are shown below.

¹H-NMR (CDCl₃=7.26 ppm) δ (ppm)=1.29 (18H, s), 1.43 (18H, s), 4.31 (4H, s), 4.78 (2H, s), 6.94 (4H, d), 7.04 (2H, s), 7.16 (4H, d), 7.32 (2H, s)

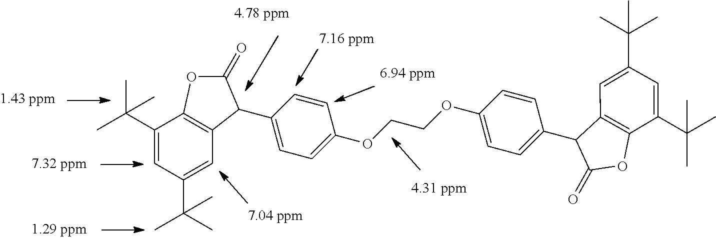 [SMILES: CC(C)(C)c1cc2c(c(C(C)(C)C)c1)OC(=O)C2c1ccc(OCCOc2ccc(C3C(=O)Oc4c3cc(C(C)(C)C)cc4C(C)(C)C)cc2)cc1]

Evaluation of Radical Trapping Performance

The compound (1) was added to a polycarbonate resin at a concentration of 150 ppm and dri-mixed for 10 minutes, and then using a twin-screw extruder (TEX30α (L/D=42, Φ=30 mm) manufactured by The Japan Steel Works, LTD.), the mixture was kneaded at a melting temperature of 260° C. to obtain pellets.

Using the obtained pellets, a plate-shaped test piece having a width of 50 mm, a length of 90 mm, and a thickness of 1.5 mm was produced with an injection molding machine (J100 EII-P manufactured by The Japan Steel Works, LTD.) under a condition of 290° C.

Next, the test piece was irradiated with ultraviolet rays (UV), and the radical generation amount was quantified by electron spin resonance (ESR).

As a result, an increase in ESR signal intensity at B=3400 G after irradiation for 1200 seconds was 0.10×10^{∧}3[a.u.].

### Example 2

A reaction was carried out by the same method as in Example 1 to obtain the following compound, except that 4.41 parts by mass of ethylene glycol diphenyl ether (manufactured by Tokyo Chemical Industry Co., Ltd.) was changed to 4.98 parts by mass of 1,2-bis(3-methylphenoxy)ethane (manufactured by SANKOSHA CO., LTD.).

Next, the radical generation amount of a test piece on a plate was quantified by the same method as in Example 1. An increase in ESR signal intensity at B=3400 G after irradiation for 1200 seconds was 0.15×10^{∧}3 [a.u.].

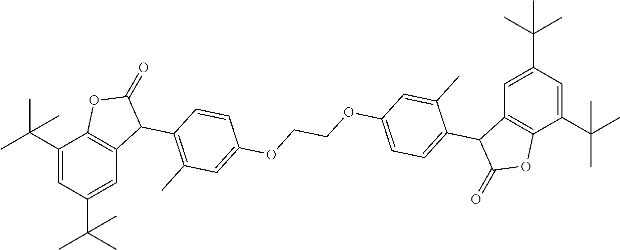 [SMILES: Cc1cc(OCCOc2ccc(C3C(=O)Oc4c3cc(C(C)(C)C)cc4C(C)(C)C)c(C)c2)ccc1C1C(=O)Oc2c1cc(C(C)(C)C)cc2C(C)(C)C]

### Example 3

A reaction was carried out by the same method as in Example 1 to obtain the following compound, except that 4.41 parts by mass of ethylene glycol diphenyl ether (manufactured by Tokyo Chemical Industry Co., Ltd.) was changed to 5.64 parts by mass of 1,2-bis(2-methoxyphenoxy)ethane synthesized according to Journal of Molecular Structure, 2019, vol. 1175, pp. 414 to 427.

Next, the radical generation amount of a test piece on a plate was quantified by the same method as in Example 1. An increase in ESR signal intensity at B=3400 G after irradiation for 1200 seconds was 0.15×10^{∧}3 [a.u.].

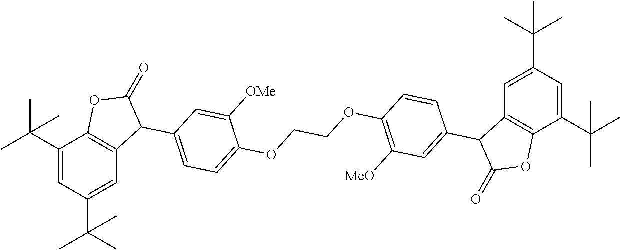 [SMILES: COc1cc(C2C(=O)Oc3c2cc(C(C)(C)C)cc3C(C)(C)C)ccc1OCCOc1ccc(C2C(=O)Oc3c2cc(C(C)(C)C)cc3C(C)(C)C)cc1OC]

### Example 4

A reaction was carried out by the same method as in Example 1 to obtain the following compound, except that 4.41 parts by mass of ethylene glycol diphenyl ether (manufactured by Tokyo Chemical Industry Co., Ltd.) was changed to 4.12 parts by mass of diphenoxymethane (manufactured by Tokyo Chemical Industry Co., Ltd.).

Next, the radical generation amount of a test piece on a plate was quantified by the same method as in Example 1. An increase in ESR signal intensity at B=3400 G after irradiation for 1200 seconds was 0.20×10^{∧}3 [a.u.].

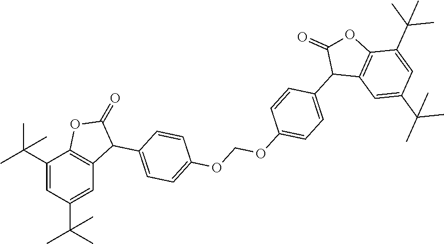 [SMILES: CC(C)(C)c1cc2c(c(C(C)(C)C)c1)OC(=O)C2c1ccc(OCOc2ccc(C3C(=O)Oc4c3cc(C(C)(C)C)cc4C(C)(C)C)cc2)cc1]

### Example 5

A reaction was carried out by the same method as in Example 1 to obtain the following compound, except that 4.41 parts by mass of ethylene glycol diphenyl ether (manufactured by Tokyo Chemical Industry Co., Ltd.) was changed to 4.69 parts by mass of 1,3-diphenoxypropane synthesized according to Journal of Molecular Structure, 2019, vol. 1175, pp. 414 to 427.

Next, the radical generation amount of a test piece on a plate was quantified by the same method as in Example 1. An increase in ESR signal intensity at B=3400 G after irradiation for 1200 seconds was 0.20×10^{∧}3[a.u.].

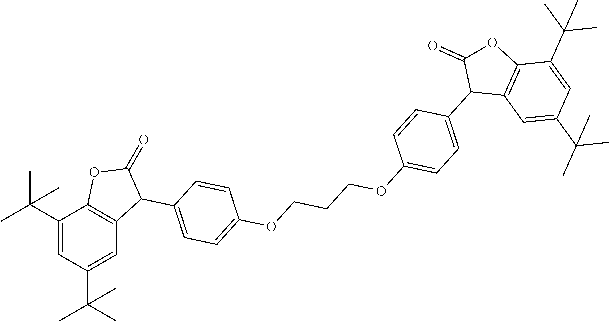 [SMILES: CC(C)(C)c1cc2c(c(C(C)(C)C)c1)OC(=O)C2c1ccc(OCCCOc2ccc(C3C(=O)Oc4c3cc(C(C)(C)C)cc4C(C)(C)C)cc2)cc1]

### Example 6

A reaction was carried out by the same method as in Example 1 to obtain the following compound, except that 4.41 parts by mass of ethylene glycol diphenyl ether (manufactured by Tokyo Chemical Industry Co., Ltd.) was changed to 5.56 parts by mass of 1,6-diphenoxyhexane synthesized according to Journal of Molecular Structure, 2019, vol. 1175, pp. 414 to 427.

Next, the radical generation amount of a test piece on a plate was quantified by the same method as in Example 1. An increase in ESR signal intensity at B=3400 G after irradiation for 1200 seconds was 0.25×10^{∧}3 [a.u.].

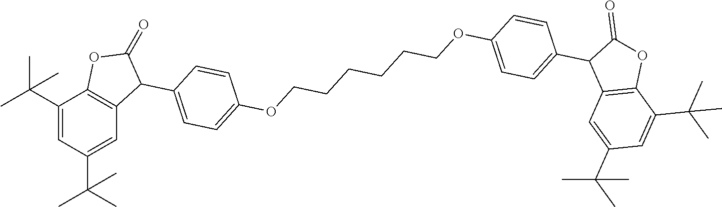 [SMILES: CC(C)(C)c1cc2c(c(C(C)(C)C)c1)OC(=O)C2c1ccc(OCCCCCCOc2ccc(C3C(=O)Oc4c3cc(C(C)(C)C)cc4C(C)(C)C)cc2)cc1]

### Example 7

A reaction was carried out by the same method as in Example 1 to obtain the following compound, except that 4.41 parts by mass of ethylene glycol diphenyl ether (manufactured by Tokyo Chemical Industry Co., Ltd.) was changed to 6.72 parts by mass of 1,10-diphenoxydecane synthesized according to Journal of Molecular Structure, 2019, vol. 1175, pp. 414 to 427.

Next, the radical generation amount of a test piece on a plate was quantified by the same method as in Example 1. An increase in ESR signal intensity at B=3400 G after irradiation for 1200 seconds was 0.30×10^{∧}3 [a.u.].

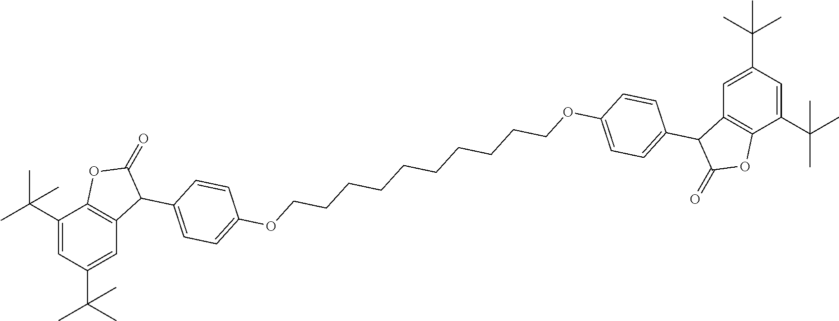 [SMILES: CC(C)(C)c1cc2c(c(C(C)(C)C)c1)OC(=O)C2c1ccc(OCCCCCCCCCCOc2ccc(C3C(=O)Oc4c3cc(C(C)(C)C)cc4C(C)(C)C)cc2)cc1]

### Example 8

A reaction was carried out by the same method as in Example 1 to obtain the following compound, except that 4.41 parts by mass of ethylene glycol diphenyl ether (manufactured by Tokyo Chemical Industry Co., Ltd.) was changed to 4.08 parts by mass of dibenzyl ether (manufactured by Tokyo Chemical Industry Co., Ltd.).

Next, the radical generation amount of a test piece on a plate was quantified by the same method as in Example 1. An increase in ESR signal intensity at B=3400 G after irradiation for 1200 seconds was 0.80×10^{∧}3 [a.u.].

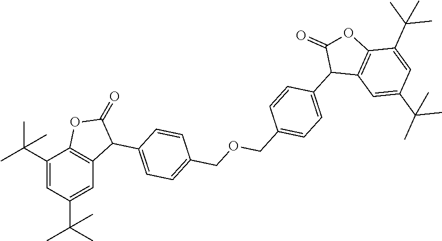 [SMILES: CC(C)(C)c1cc2c(c(C(C)(C)C)c1)OC(=O)C2c1ccc(COCc2ccc(C3C(=O)Oc4c3cc(C(C)(C)C)cc4C(C)(C)C)cc2)cc1]

### Example 9

A reaction was carried out by the same method as in Example 1 to obtain the following compound, except that 21.3 parts by mass of 2,4-di-tert-butylphenol (manufactured by Tokyo Chemical Industry Co., Ltd.) was changed to 17.0 parts by mass of 2-tert-butyl-p-cresol (manufactured by Tokyo Chemical Industry Co., Ltd.).

Next, the radical generation amount of a test piece on a plate was quantified by the same method as in Example 1. An increase in ESR signal intensity at B=3400 G after irradiation for 1200 seconds was 0.12×10^{∧}3 [a.u.].

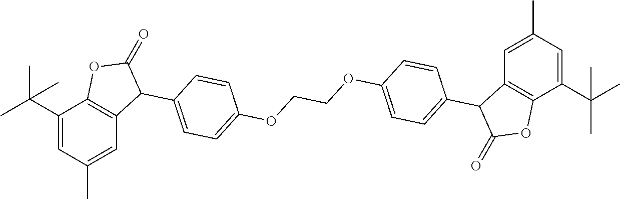 [SMILES: Cc1cc2c(c(C(C)(C)C)c1)OC(=O)C2c1ccc(OCCOc2ccc(C3C(=O)Oc4c3cc(C)cc4C(C)(C)C)cc2)cc1]

### Example 10

A reaction was carried out by the same method as in Example 1 to obtain the following compound, except that 21.3 parts by mass of 2,4-di-tert-butylphenol (manufactured by Tokyo Chemical Industry Co., Ltd.) was changed to 24.2 parts by mass of 2,4-di-tert-amylphenol (manufactured by Tokyo Chemical Industry Co., Ltd.).

Next, the radical generation amount of a test piece on a plate was quantified by the same method as in Example 1. An increase in ESR signal intensity at B=3400 G after irradiation for 1200 seconds was 0.10×10^{∧}3 [a.u.].

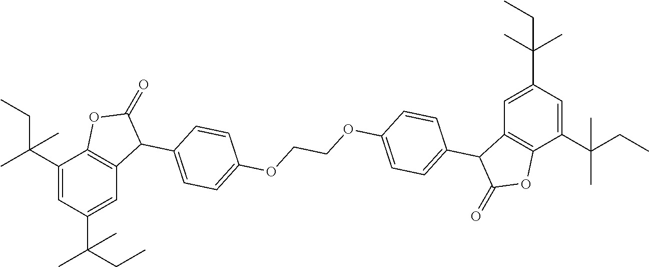 [SMILES: CCC(C)(C)c1cc2c(c(C(C)(C)CC)c1)OC(=O)C2c1ccc(OCCOc2ccc(C3C(=O)Oc4c3cc(C(C)(C)CC)cc4C(C)(C)CC)cc2)cc1]

### Example 11

A reaction was carried out by the same method as in Example 1 to obtain the following compound, except that 21.3 parts by mass of 2,4-di-tert-butylphenol (manufactured by Tokyo Chemical Industry Co., Ltd.) was changed to 21.3 parts by mass of 4-(1,1,3,3-tetramethylbutyl)phenol (manufactured by Tokyo Chemical Industry Co., Ltd.).

Next, the radical generation amount was quantified by the same method as in Example 1. An increase in ESR signal intensity at B=3400 G after irradiation for 1200 seconds was 0.10×10^{∧}3 [a.u.].

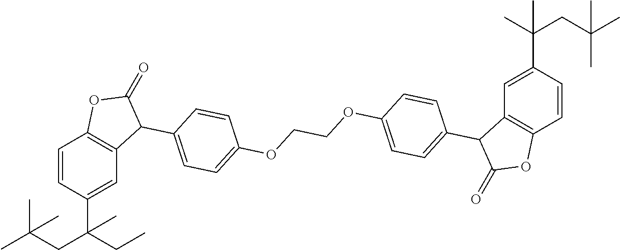 [SMILES: CCC(C)(CC(C)(C)C)c1ccc2c(c1)C(c1ccc(OCCOc3ccc(C4C(=O)Oc5ccc(C(C)(C)CC(C)(C)C)cc54)cc3)cc1)C(=O)O2]

### Example 12

A reaction was carried out by the same method as in Example 1 to obtain the following compound, except that 21.3 parts by mass of 2,4-di-tert-butylphenol (manufactured by Tokyo Chemical Industry Co., Ltd.) was changed to 28.5 parts by mass of 4-dodecyl-o-cresol (manufactured by Tokyo Chemical Industry Co., Ltd.).

Next, the radical generation amount was quantified by the same method as in Example 1. An increase in ESR signal intensity at B=3400 G after irradiation for 1200 seconds was 0.12×10^{∧}3 [a.u.].

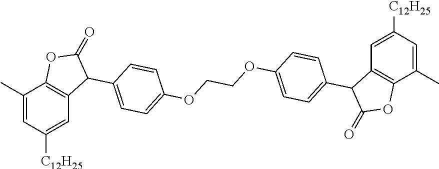

### Example 13

A reaction was carried out by the same method as in Example 1 to obtain the following compound, except that 21.3 parts by mass of 2,4-di-tert-butylphenol (manufactured by Tokyo Chemical Industry Co., Ltd.) was changed to 14.9 parts by mass of 1-naphthol (manufactured by Tokyo Chemical Industry Co., Ltd.).

Next, the radical generation amount was quantified by the same method as in Example 1. An increase in ESR signal intensity at B=3400 G after irradiation for 1200 seconds was 0.22×10^{∧}3 [a.u.].

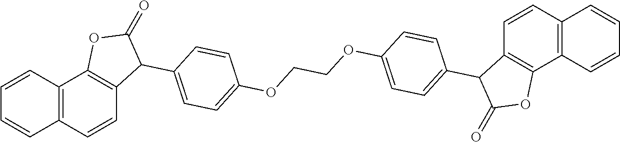 [SMILES: O=C1Oc2c(ccc3ccccc23)C1c1ccc(OCCOc2ccc(C3C(=O)Oc4c3ccc3ccccc43)cc2)cc1]

### Example 14

A plate-shaped test piece was obtained by the same operation as in Example 1, except that the polycarbonate resin was changed to a polyester resin.

Next, the radical generation amount was quantified by the same method as in Example 1. An increase in ESR signal intensity at B=3400 G after irradiation for 1200 seconds was 0.07×10^{∧}3 [a.u.].

### Example 15

A plate-shaped test piece was obtained by the same operation as in Example 1, except that, under the conditions of Example 1, UV-008 (manufactured by FUJIFILM Corporation) was further added to the polyester resin at a concentration of 150 ppm.

Next, the radical generation amount was quantified by the same method as in Example 9. An increase in ESR signal intensity at B=3400 G after irradiation for 1200 seconds was 0.05×10^{∧}3 [a.u.].

### Comparative Example 1

The radical generation amount was quantified by the same method as in Example 1, except that the compound (1) was not added.

As a result, an increase in ESR signal intensity at B=3400 G after irradiation for 1200 seconds was 1.50×10^{∧}3[a.u.].

### Comparative Example 2

The radical generation amount was quantified by the same method as in Example 1, except that, instead of 150 ppm of the compound (1), 150 ppm of a compound (H1) represented by Formula (H1) was used.

As a result, an increase in ESR signal intensity at B=3400 G after irradiation for 1200 seconds was 1.20×10^{∧}3[a.u.].

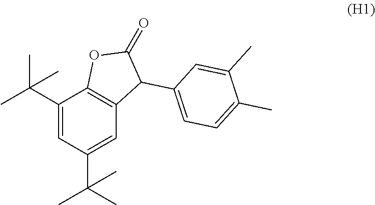 [SMILES: Cc1ccc(C2C(=O)Oc3c2cc(C(C)(C)C)cc3C(C)(C)C)cc1C]

From the results shown in Examples 1 to 15 and Comparative Examples 1 and 2, in a case where the lactone compound represented by Formula (I) described above was used, compared to a case where the lactone compound was not used or a case where a lactone compound which did not correspond to the Formula (I) described above was used, since the increase in ESR signal intensity was half or less, it was found that the compound represented by Formula (I) described above was a compound having excellent radical trapping performance.

In particular, in a case where the compound in which L in Formula (I) described above was represented by L1, compared to a case where the lactone compound was not used or a case where a lactone compound which did not correspond to the Formula (I) described above was used, since the increase in ESR signal intensity was 1/10 or less, it was found to be a compound having more excellent radical trapping performance.

What is claimed is:

1. A lactone compound represented by Formula (I), 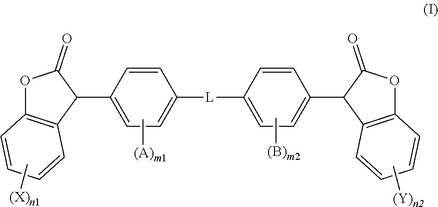
here, in Formula (I),
A, B, X, and Y each independently represent an alkyl group or an alkoxy group,
m1 represents an integer of 0 to 4, and in a case where m1 is an integer of 2 to 4, a plurality of A's may be the same substituent or substituents different from each other,
m2 represents an integer of 0 to 4, and in a case where m2 is an integer of 2 to 4, a plurality of B's may be the same substituent or substituents different from each other,
n1 represents an integer of 0 to 4, and in a case where n1 is an integer of 2 to 4, a plurality of X's may be the same substituent or substituents different from each other and may be bonded to each other to form a ring,
n2 represents an integer of 0 to 4, and in a case where n2 is an integer of 2 to 4, a plurality of Y's may be the same substituent or substituents different from each other, and may be bonded to each other to form a ring, and
L represents a linking group represented by Formula (L1) or Formula (L2), 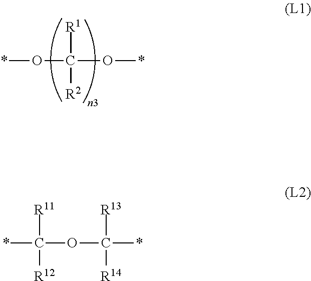
here, in Formulae (L1) and (L2), * represents a bonding position to a phenylene group in Formula (I),
in Formula (L1), R¹ and R² each independently represent a hydrogen atom, an alkyl group, or an alkoxy group, n3 represents an integer of 1 to 12, and in a case where n3 is an integer of 2 to 12, a plurality of R's may be the same substituent or substituents different from each other, and a plurality of R²'s may be the same substituent or substituents different from each other, and
in Formula (L2), R¹¹, R¹², R¹³, and R¹⁴ each independently represent a hydrogen atom, an alkyl group, or an alkoxy group.

2. The lactone compound according to claim 1,
wherein both m1 and m2 in Formula (I) represent 0.

3. The lactone compound according to claim 1,
wherein both n1 and n2 in Formula (I) represent 2.

4. The lactone compound according to claim 2,
wherein both n1 and n2 in Formula (I) represent 2.

5. The lactone compound according to claim 3,
wherein the lactone compound is represented by Formula (I-1), 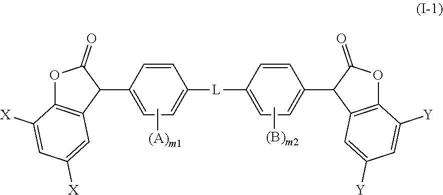
here, in Formula (I-1),
A, B, X, and Y each independently represent an alkyl group or an alkoxy group,
m1 represents an integer of 0 to 4, and in a case where m1 is an integer of 2 to 4, a plurality of A's may be the same substituent or substituents different from each other,
m2 represents an integer of 0 to 4, and in a case where m2 is an integer of 2 to 4, a plurality of B's may be the same substituent or substituents different from each other, and
L represents a linking group represented by Formula (L1) or Formula (L2), 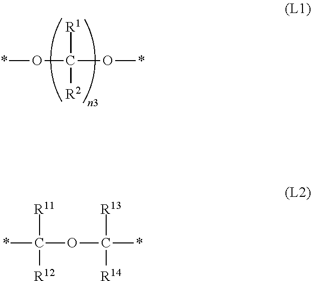
here, in Formulae (L1) and (L2), * represents a bonding position to a phenylene group in Formula (I-1),
in Formula (L1), R¹ and R² each independently represent a hydrogen atom, an alkyl group, or an alkoxy group, n3 represents an integer of 1 to 12, and in a case where n3 is an integer of 2 to 12, a plurality of R¹¹'s may be the same substituent or substituents different from each other, and a plurality of R²'s may be the same substituent or substituents different from each other, and
in Formula (L2), R¹¹, R¹², R¹³, and R¹⁴ each independently represent a hydrogen atom, an alkyl group, or an alkoxy group.

6. The lactone compound according to claim 4,
wherein the lactone compound is represented by Formula (I-1), 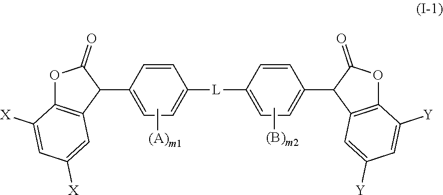
here, in Formula (I-1),
A, B, X, and Y each independently represent an alkyl group or an alkoxy group,
m1 represents an integer of 0 to 4, and in a case where m1 is an integer of 2 to 4, a plurality of A's may be the same substituent or substituents different from each other,
m2 represents an integer of 0 to 4, and in a case where m2 is an integer of 2 to 4, a plurality of B's may be the same substituent or substituents different from each other, and
L represents a linking group represented by Formula (L1) or Formula (L2), 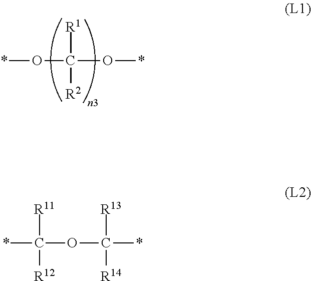
here, in Formulae (L1) and (L2), * represents a bonding position to a phenylene group in Formula (I-1),
in Formula (L1), R¹ and R² each independently represent a hydrogen atom, an alkyl group, or an alkoxy group, n3 represents an integer of 1 to 12, and in a case where n3 is an integer of 2 to 12, a plurality of R¹'s may be the same substituent or substituents different from each other, and a plurality of R²'s may be the same substituent or substituents different from each other, and
in Formula (L2), R¹¹, R¹², R¹³, and R¹⁴ each independently represent a hydrogen atom, an alkyl group, or an alkoxy group.
